(19) United States
(12) Patent Application Publication
(10) Pub. No.: US 2008/0163390 A1
(43) Pub. Date: Jul. 3, 2008

(54) METHODS AND COMPOSITIONS FOR PROVIDING SA-INDEPENDENT PATHOGEN RESISTANCE IN PLANTS

(75) Inventors: Pradeep Kachroo, Lexington, KY (US); Aardra Kachroo, Lexington, KY (US); A.C. Chandra-Shekara, River Falls, WI (US)

Correspondence Address: Teresa Stanek Rea, Crowell Moring, 1001 Pennsylvania Ave. N.W., Washington, DC, 20004, US

(73) Assignee: UNIVERSITY OF KENTUCKY RESEARCH FOUNDATION, Lexington, KY (US)

(21) Appl. No.: 11/619,574

(22) Filed: Jan. 3, 2007

(51) Int. Cl. C12N 15/82; A01H 5/00

(52) U.S. Cl. 800/276; 800/279; 800/301

(57) ABSTRACT

This disclosure relates to methods and compositions for modulating disease resistance in plants and transgenic plants comprising reducing the amount of 18:1 fatty acids.

### STATEMENT REGARDING FEDERALLY SPONSORED RESEARCH

This invention was supported in part by Grant No. MCB 0421914 awarded by the National Science Foundation and 2004-03287 awarded by the USDA-NRI. The government may have certain rights in this invention.

### TECHNICAL FIELD

This disclosure relates to methods and compositions for modulating diesease resistance in plants and transgenic plants.

### BACKGROUND

Salicylic acid (SA) has emerged as a key signal molecule in the deployment of systemic acquired resistance (SAR). After the initial observation that exogenous application of SA induces resistance in tobacco, SA has been shown to induce resistance in many plant species. Exogenous application of SA also induces expression of the same class of pathogenesis-related (PR) (PR-1, BGL2 [PR-2], and PR-5) genes as those induced during SAR. A strong correlation has been observed between the in vivo increase in SA levels in infected plants and both the expression of PR genes and development of resistance. In addition, SA appears to be involved in the activation of HR cell death and restriction of pathogen spread. The strongest evidence supporting the signaling role of SA in plant defense comes from studies on plants unable to accumulate SA upon pathogen infection. For example, transgenic tobacco and Arabidopsis plants constitutively expressing the Pseudomonas putida nahG gene, which encodes the SA-degrading enzyme salicylate hydroxylase, fail to develop SAR and are hypersusceptible to pathogen infection. Likewise, preventing SA accumulation by application of SA biosynthesis inhibitors also makes otherwise resistant Arabidopsis plants susceptible to Peronospora parasitica. Conversely, the elevated levels of SA present in the Arabidopsis acd (accelerated cell death; Greenberg et al. 1994; Rate et al. 1999), lsd (lesion simulating disease; Dietrich et al. 1994; Weymann et al. 1995), cpr (constitutive expresser of PR genes; Bowling et al., 1994, 1997; Clarke et al. 1998; Silva et al. 1999), ssi (suppressor of salicylate insensitivity of npr1-5; Shah et al. 1999), and dnd (defense with no HR cell death; Yu et al. 1998) mutants lead to constitutive expression of PR genes and SAR.

### SUMMARY

The disclosure provides a method of inducing pathogen and insect resistance in a plant comprising contacting the plant with an agent that reduces 18:1 fatty acids, wherein the reduction of 18:1 fatty acids increases the production of resistance gene transcription. In one aspect, the agent comprises a polynucleotide that reduces or inhibits the function of a stearoyl-acyl carrier protein desaturase. In another aspect, the resistance gene is selected from the group consisting of SSI4, RPS2, RPM1, RPP1, HRT and any combination thereof. In yet another aspect, the reduction or inhibition of function of the desaturase causes a reduction in 18:1 fatty acids and an SA-independent constitutive expression of PR genes.

The disclosure provides a method of inducing pathogen and insect resistance in a plant comprising contacting the plant with a composition that reduces or inhibits the production of 18:1 and causes increased transcription of resistance genes. The compsition can comprise glycerol or a glycerol derivative. In another aspect, the composition comprises glycerol or a glycerol derivative and salicylic acid. The composition can comprise glycerol or a glycerol derivative and an agent that increases ACT1 activity.

The disclosure also provides a method of increasing HRT (resistance) gene expression in a plant comprising inhibiting the production of 18:1.

The disclosure also provides a transgenic plant that is resistant to a pathogen and tolerant to insect infestation comprising reduced expression of SSI2, wherein the plant comprises reduced 18:1.

The disclosure futher provides a method to enhance resistance of a plant to plant pathogens or other disease causing agents and insects comprising decreasing production or activity of a SSI2 gene product in the plant thereby reducing 18:1 in the plant.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-D shows ssi2- and cpr5-mediated resistance signaling in various double mutant backgrounds. (A) Percentage TCV resistant wild type (HRT or hrt) and single and double mutant plants in ssi2 and cpr5 backgrounds. The numbers of plants tested are indicated above each bar. All plants were analyzed 3-weeks post-inoculation. Asterisk indicates 100% susceptibility. (B) RT-PCR analyses showing HRT transcript levels in indicated genotypes. The level of β-tubulin was used as an internal control to normalize the amount of cDNA template. (C) Typical morphological phenotypes of TCV-inoculated plants; susceptible plants showed crinkling, stunted bolt development and drooping of bolts. The resistant plants were morphologically similar to the mock-inoculated plants. (D) Systemic spread of TCV to uninoculated tissue in TCV-inoculated plants. RNA was extracted from the uninoculated tissues at 18 dpi and analyzed for the presence of the viral transcripts (TCV-U). Ethidium bromide staining of rRNA was used as a loading control.

FIG. 2A-D shows HRT transcript levels, TCV resistance and viral replication in leaves containing normal or higher oleate levels. (A) RT-PCR analyses showing basal level expression of HRT in HRT ssi2 act1 and HRT ssi2 gly1 plants. The level of β-tubulin was used as an internal control to normalize the amount of cDNA template. The 18:1 levels are a mean of six independent replicates. (B) Systemic spread of TCV to uninoculated tissue in TCV-inoculated plants. RNA was extracted from the uninoculated tissues at 18 dpi and analyzed for the presence of the viral transcripts (TCV-U). Ethidium bromide staining of rRNA was used as a loading control. (C) Typical morphological phenotypes of mock-and TCV-inoculated HRT ssi2 act1 and HRT ssi2 gly1 plants. The susceptible plants showed crinkling, stunted bolt development and drooping of bolts. Plants were photographed at 8 dpi. (D) Effect of 18:1 infiltrations on viral replication in the inoculated leaf. Oleic acid (O, 1 mM) or water (W) was injected 24 h before or after (last two lanes) TCV inoculation and the samples were harvested 3 days post inoculation (dpi).

FIG. 3A-F shows PR-1 and R gene expression levels, HR formation and TCV resistance in plants treated either with water or glycerol. (A) PR-1 gene expression and 18:1 content in water (W)- and glycerol (G)-treated plants. Ethidium bromide staining of rRNA was used as a loading control. The 18:1 levels are a mean of six independent replicates. (B) RT-PCR analyses showing expression of HRT and SSI4 genes in water- and glycerol-treated plants shown in A. The level of β-tubulin was used as an internal control to normalize the amount of cDNA template. (C) Visible HR formation in water (W)- and glycerol (G)-treated HRT sid2, HRT pad4 and HRT act1 plants at 3 dpi. (D) Percentage TCV resistant plants obtained after exogenous application of water, SA or glycerol. Wild type (HRT or hrt), HRT sid2, HRT pad4 and HRT act1 plants were inoculated 48 h post-treatment and resistance was analyzed 3-weeks post-inoculation. The number of plants tested are indicated above each bar. Asterisk indicates 100% susceptibility. (E) Systemic spread of TCV to uninoculated tissue in TCV-inoculated plants. RNA was extracted from the uninoculated tissues at 18 dpi and analyzed for the presence of the viral transcripts (TCV-U). Ethidium bromide staining of rRNA was used as a loading control. (F) Typical morphological phenotypes of TCV-inoculated plants that were sprayed either with water (W) or glycerol (G) prior to inoculation. Plants were photographed at 18 dpi.

FIG. 4A-C shows oleic acid-modulated expression of R genes and resistance to bacterial pathogen. (A) RT-PCR analysis of various R genes in wild type (HRT), HRT ssi2 and HRT ssi2 sid2 backgrounds. The level of β-tubulin was used as an internal control to normalize the amount of cDNA template. (B) RT-PCR analysis of various R genes in wild type (HRT), HRT cpr5 and HRT cpr5 sid2 backgrounds. The level of β-tubulin was used as an internal control to normalize the amount of cDNA template. (C) Growth of P. syringae on wild-type (HRT or hrt), HRT ssi2, HRT sid2 and HRT ssi2 sid2 leaves. The Nössen ecotype was also tested and showed resistance similar to that seen in Di-17 and Col-0 plants. Four leaf discs were harvested from infected leaves 3 days post inoculation, ground in 10 mM MgCl2, and the bacterial numbers tittered. The bacterial numbers ±SD (n=4) are presented as log of colony-forming units (CFU) per cm2. The experiment was independently performed twice with similar results.

FIG. 5A-B shows Oleic acid- and stearoyl-ACP-desaturase transcript-levels after mock- and TCV-inoculation. (A) The inoculated leaves from Di-17 plants were sampled at 0-72 h post inoculation and processed for fatty acid levels. The values are a mean of six independent replicates. The error bars represent SD. (B) Stearoyl-ACP-desaturase (S-ACP-DES) transcript levels in mock- and TCV-inoculated plants 72 h post inoculation. Data from two independently extracted RNA samples are shown here. In addition to SSI2, the Arabidopsis genome encodes six S-ACP-DES-like enzymes. Besides SSI2, four other isozymes are capable of desaturating 18:0-ACP (S-ACP-DES1, S-ACP-DES3, S-ACP-DES4, S-ACP-DES5) but with greatly reduced specific activities. In comparison to SSI2, the specific activities of S-ACP-DES3, and 5 are ˜219- and ˜45-fold less on C18:0-ACP substrate and ˜1.6- and ˜29-fold less on C16:0-ACP substrate, respectively. However, overexpression of the S-ACP-DES1 isoform, which has ˜53-fold less specific activity on C18:0-ACP as compared to SSI2, in ssi2 plants results in restoration of 18:1 levels and thereby rescues all ssi2-associated phenotypes. Thus, increased expression of a low specific activity S-ACP-DES is sufficient to compensate for a mutation in ssi2. Transcript levels of S-ACP-DES1, 2, 4 and 6 were either not detected or showed no difference between mock- and TCV-inoculated plants.

FIG. 6A-B depicts an abbreviated scheme for lipid biosynthesis and in plant oleic acid levels after 18:1 infiltrations. (A) De novo fatty acid (FA) synthesis occurs exclusively in the plastids of all plant cells and leads to the synthesis of palmitic acid (16:0)-acyl carrier protein (ACP) and oleic acid (18:1)-ACP. These FAs enter glycerolipid synthesis either via the prokaryotic pathway in the inner envelope of chloroplasts or are exported out of plastids as CoA thioesters to enter the eukaryotic glycerolipid synthesis pathway. Symbols for various components are: CoA, coenzyme A; Lyso-PA, acyl-G3P; PA, phosphatidic acid; PG, phosphatidylglycerol; MGDG, monogalactosyldiacylglycerol; DGDG, digalactosyldiacylglycerol; SL, sulfolipid; DAG, diacylglycerol; DHAP, dihydroxyacetone phosphate. (B) In planta oleic acid levels after 18:1 infiltrations. The HRT ssi2 sid2 leaves were infiltrated with water or 1 mM oleic acid and the fatty acid levels were determined 24 h post treatment from treated and untreated leaves. The 18:1 levels from the untreated wild type plant (HRT) were used as a control.

### DETAILED DESCRIPTION

As used herein and in the appended claims, the singular forms “a,” “and,” and “the” include plural referents unless the context clearly dictates otherwise. Thus, for example, reference to “a compound” includes a plurality of such compounds and reference to “the plant” includes reference to one or more plants known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Recognition of a pathogen by a plant triggers a cascade of responses in plants. Recognition involves strain-specific detection of a pathogen-encoded elicitor, through direct or indirect interaction, with the corresponding resistance (R) gene product. Such an interaction (also known as incompatible interaction) triggers one or more defense signaling cascades and is often associated with induction of hypersensitive response (HR) at the site of pathogen entry. HR is one of the first visible manifestations of the host-induced defense response and is thought to help prevent multiplication and movement by confining the pathogen to dead cells. An R gene-mediated recognition of pathogen can also lead to the accumulation of various phytohormones including SA, jasmonic acid (JA) and/or ethylene, which in turn signal activation of defense gene expression. Each hormone activates a specific pathway wherein the genes act individually, synergistically or antagonistically, depending upon the pathogen involved, and a combined effect of which confers resistance and prevents spread of the pathogen to uninoculated parts of the plants.

Lipids are essential in the composition of all plant cells. Although plant lipids cover a wide range of compounds, the majority of lipids are derived from two important metabolic pathways, the fatty acid biosynthetic pathway and the glycerolipid biosynthetic pathway. Plants naturally produce an assortment of fatty acids which they incorporate into a wide assortment of lipids which perform different functions. Polar glycerolipids (phospholipids and glycolipids), for example, contain two fatty acids attached to both sn-1 and sn-2 positions of the glycerol backbone and a polar headgroup attached to the sn-3 position. Polar glycerolipids play an essential role in cell membrane structure and function. Triacylglycerols, on the other hand, have all three positions of the glycerol backbone esterified with fatty acids and are the major storage lipids in oil-producing plant tissues, such as in plant seeds, and are usually known as plant oils.

The specific properties of a plant oil are dependent on the fatty acid composition of the oil, which in turn affects the nutritional quality of the oil. The health value of high levels of monounsaturates, particularly oleic acid, as the major dietary fat constituent has been established by recent studies. For example, canola oil, which typically contains at least 60% oleic acid (c18:1, Δ⁹), has been proven effective in lowering cholesterol in human blood. It has also been shown, however, that high levels of all monounsaturated fatty acids are not necessarily beneficial. For example, it has been suggested that palmitoleic acid (c16:1, Δ⁹) may have certain health disadvantages, such as behaving as a saturated fatty acid in its effect on cholesterol (Nestel et al., 1994, J Lipid Res 35(4):656-662) effecting atrioventicular conduction in the heart (Dhein et al, 1999, Br. J. Pharmacol 128(7) 1375-1384) and correlating with high blood pressure in men at high risk of coronary heart disease (Simon et al., Hypertension Feb. 27, 1996 (2):303-7). As a result, because of these medical and nutritional effects, there is an interest in lowering the level of saturated fatty acids in plant oils beyond certain limits (the limit of allowable saturated fatty acid proportions in canola oil, for example, is 7%).

The fatty acid composition of plant oils is determined both by the genotype of the plant and the plant's response to environmental factors such as light, temperature and moisture. Genetic modification by plant breeding or genetic engineering may be used to modify fatty acid metabolic pathways and thereby modify plant oil composition.

In plants, fatty acids are generally synthesized in the plastid or chloroplast by the FAS system in which the elongating chain is generally esterified to acyl-carrier protein (ACP) as palmitic acid (16:0) and stearic acid (18:0) esterified to ACP (i.e., 16:0-ACP and 18:0-ACP, respectively). A known soluble plant stearoyl-ACP Δ⁹ desaturase enzyme is located in the chloroplast where it is understood to catalyze the conversion of stearoyl-ACP (18:0-ACP) to oleoyl-ACP (18:1-ACP). These acyl-ACPs may either be used for glycerolipid synthesis in the chloroplast or transported out of chloroplast into the cytoplasm as acyl-CoAs. It is generally believed that the stearoyl-ACP Δ⁹ enzyme is the only soluble plant desaturase, so that palmitic acid and stearic acid exported from the chloroplast will not undergo further desaturation. Therefore, the level of saturation is largely determined by the amount of saturated fatty acids exported out of the chloroplast and into the cytoplasm.

Resistance (R) gene-dependent defense signaling in plants is often mediated by the plant hormone salicylic acid (SA) and requires the function of several downstream components including EDS1 and PAD4. In Arabidopsis, resistance to Turnip Crinkle Virus (TCV) is dependent upon the R gene, HRT, high levels of SA and a functional PAD4 protein. The disclosure provides an alternate, oleic acid (18:1)-mediated pathway that regulates defense signaling by upregulating R gene expression. Reduction in 18:1 induces HRT expression and confers resistance to TCV in a SA- and PAD4-independent manner. This low 18:1-derived signal induces the expression of multiple, structurally divergent, R genes, resulting in broad-spectrum disease resistance. Although, SA-dependent and 18:1-regulated pathways had additive effects, the 18:1-regulated pathway was more effective in conferring resistance in various susceptible backgrounds. Inoculation with TCV, however, did not activate the 18:1-regulated pathway in HRT plants, instead it resulted in the induction of several genes that encode 18:1-synthesizing isozymes and consequently 18:1 levels remained constant during a resistance response. This suggests that the 18:1-regulated pathway is specifically targeted during pathogen infection and that alterations of 18:1 levels serves as a novel strategy for promoting broad-spectrum disease resistance.

The disclosure demonstrates that fatty acid levels regulate expression of HRT and several other structurally different R genes. For example, methods and compositions that reduce oleic acid levels results in an increase in transcript levels of HRT and confers resistance to TCV via an alternative pathway. This alternative pathway does not require SA or other defense components that are generally required for resistance to TCV.

Several components of the SA-mediated pathway have been identified and mutations in these pathway components lead to enhanced susceptibility to various pathogens. Mutations in eds1 (a putative lipase), eds5 (a member of the MATE transporter family), pad4 (a putative lipase) and sid2, lower or abolish pathogen-induced increase in SA levels. The EDS1, EDS5, PAD4 and SID2 proteins participate in both basal resistance to virulent pathogens as well as R protein-mediated response to avirulent pathogens. Resistance signaling mediated via a majority of R proteins that contain Toll-interleukin1-like (TIR) domains at their N-terminal end is dependent on EDS1. However, a few R proteins containing coiled coil (CC) domains at their N-terminal end are also dependent on EDS1. This includes HRT, which confers resistance to Turnip Crinkle Virus and RPW8, which confers broad-spectrum resistance to powdery mildew. However, RPW8 is not a nucleotide binding (NB)-leucine rich repeat (LRR) type R protein, instead it contains a N-terminal transmembrane domain in addition to the CC domain.

Besides EDS1, resistance to TCV in Arabidopsis is also dependent on EDS5, PAD4 and SID2 genes of the SA pathway. Resistance to TCV also requires a recessive locus, rrt. Induced expression of HRT via exogenous application of SA or transgenic overexpression overcomes the requirement for rrt and dramatically reduces HR lesion size. Even though SA is downstream to the initial recognition and signaling events, its exogenous application is only able to confer resistance in plants that contain at least one copy of HRT and a functional PAD4 protein. Mobilizing HRT in susceptible mutant backgrounds carrying high SA, such as ssi2 or cpr5, can constitutively increase expression of HRT and resistance to TCV. The ssi2 and cpr5 plants contain mutations in genes encoding for stearoyl-acyl carrier protein desaturase and a transmembrane protein of unknown function, respectively. While molecular mechanisms responsible for constitutive defense in cpr5 are not clear, characterization of ssi2 and its suppressors has implicated low levels of oleic acid (18:1) as responsible for altered defense signaling. The diverse biology of ssi2 and cpr5 mutants suggests that high levels of endogenous SA may be responsible for the upregulation of HRT in HRT ssi2 and HRT cpr5 plants.

Accordingly, the disclosure provides methods and compositions that provide an alternative pathway for disease resistance independent of SA-induced resistance. Such methods and compositions are useful to broaden disease resistance in plant types (e.g., plant genotypes) that comprise a mutant SA-mediated resistance pathway resulting in a dysfunctional disease resistance. In addition, wherein there exists and active SA-mediated resistance pathway, the inhibition of fatty acid synthesis (i.e., 18:1) results in an additive, more effective, disease resistance in plants.

The term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the method of the disclosure is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae.

With respect to genotypes of the disclosure, the terms “SSI2” and “ssi2” are used. The term “SSI2” is used to designate the naturally-occurring or wild-type genotype. This genotype has the phenotype of the naturally-occurring spectrum of disease resistance and susceptibility. The term “ssi2” refers to a genotype having recessive mutation(s) in the wild-type SSI2 gene. The phenotype of ssi2 individuals results in enhanced resistance to selected plant pathogens. The term “SSI2” refers to the protein product of the SSI2 gene.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence or a modulating nucleic acid (e.g., an antisense, an siRNA or ribozyme) operably linked (i.e., under regulatory control of) to an appropriate inducible or constitutive regulatory sequences that allow for the expression of a polypeptide or modulating nucleic acid. The expression cassette can be introduced into a plant by transformation or by breeding after transformation of a parent plant. Such methods can be used in a whole plant, including seedlings and mature plants, as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

In Arabidopsis thaliana, the SSI2 gene is located on chromosome 2, approximately 0.2 cM from the AthB102 marker on the centromeric side and 3.7 cM from GBF on the telomeric side. Recombination analysis with these markers placed ssi2 within a 41 kb region encompassed by the bacterial artificial chromosome (BAC) F18019 (Genbank Accession No. AC002333). Four open reading frames were identified within a corresponding 11.7 kb sub-region in clone F23 of a transformation competent artificial chromosome (TAC) library. Open reading frame (ORF) 2 was determined to be SSI2. The nucleotide and polypeptide sequences of SSI2 are known (see, e.g., GenBank Accession No. NM_{—}180069, incorporated herein by reference. Homologs from other species are also encompassed by the disclosure. Such homologs will have at least 60%, 70%, 80%, 90%, 95%, 98% or 99% identity to the nucleic acid or polypeptides sequence of as set forth in GenBank Accession No. NM_{—}180069 and encode a polypeptide having stearoyl-acyl carrier protein desaturase activity. Polynucleotide (RNA, DNA, or nucleic acid analogs-based molecules) that hybridize to polynucleotide comprising the sequence as set forth in GenBank Accession No. NM_{—}180069 and encoding a polypeptide having stearoyl-acyl carrier protein desaturase activity are also encompassed by the disclosure. For example, the silencing of ssi2 homologs in Soybean plants (using methods described herein) results in inhbition of 18:1 production an dleads to induction of defense responses, similar to those seen in Arabidopsis.

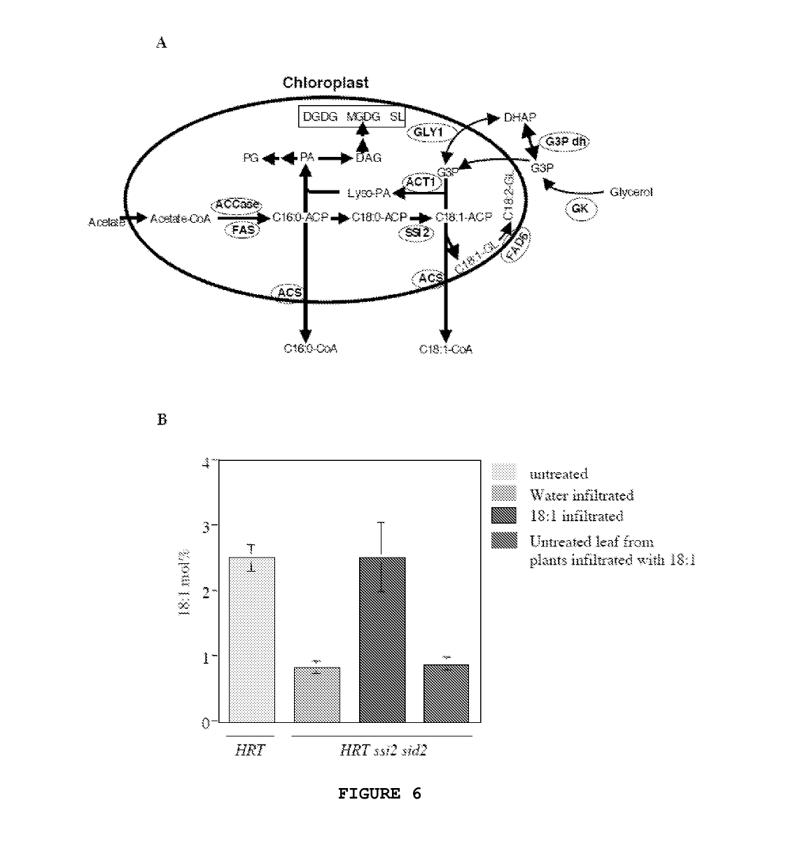

SSI2 encodes a fatty acid desaturase, of which the archetype is the stearoyl-ACP desaturase (S-ACP-DES). This enzyme is a Δ⁹ fatty acid desaturase that desaturates stearoyl-ACP (18:0 ACP). The encoded SSI2 enzyme has a specific activity and substrate preference (88:1 for 18-versus 16-carbon chain length FA) that are characteristic of S-ACP-DES. The fatty acid composition of ssi2 mutant plants reflects a change in SSI2 activity by exhibiting elevated 18:0 and decreased 18:1 fatty acid levels, as compared with wildtype plants. Referring to FIG. 6, there is shown a biosynthetic pathway. Desaturation of stearic acid (18:0)-ACP to 18:1-ACP catalyzed by the SSI2/FAB2 encoded stearoyl-ACP desaturase, is one of the key steps in the FA biosynthesis pathway that regulates levels of unsaturated FAs in the cell. The 18:1-ACP generated in this reaction enters the prokaryotic pathway through acylation of glycerol-3-phosphate (G3P) and this reaction is catalyzed by the ACT1-encoded G3P acyltransferase. G3P can be made via a cytosolic enzyme glycerol kinase (GK) or via G3P dehydrogenase (G3Pdh, encoded by GLY1). Desaturation of 18:1 to 18:2 and 18:3 on membrane glycerolipids (GL) is catalyzed by FAD6 and FAD7/FAD8 encoded desaturases, respectively, that are present on plastid envelop. Esterification of CoA group is mediated by acyl-CoA synthetase (ACS).

The fact that a defect in the fatty acid desaturation pathway leads to activation of certain defense responses and inhibition of others indicates that one or more FA-derived signals modulates cross-talk between different defense pathways. Since the 18:1 fatty acid pool is decreased when SSI2 activity is impaired, the fatty acid-derived signal molecule(s) comprise 18:1 fatty acids. Without intending to be limited by any explanation as to mechanism, it is possible that, the fatty acid-derived signal inhibits the SA-dependent pathway. Loss of this signal in ssi2 plants would result in constitutive activation of the SA-dependent response. The possibility that high levels of 18:0 might lead to activiation of SA responses is ruled out because several suppressors have been isolated which continue to accumulate high levels of 18:0 but are restored in their defense signaling.

The disclosure demonstrates features a FA-derived signaling molecule(s) that can be manipulated through the down-regulation of the SSI2 FA desaturase, resulting in specific modifications of plant defense responses including the upregulation of “R” gene transcription.

Due to the unique phenotype conferred by the ssi2 mutation, it is easy to screen populations of mutagenized plants (e.g., by FA profile analysis) and obtain other ssi2 mutants. Such ssi2 mutants from all other species of plants are considered to be within the scope of this disclosure.

The disclosure encompasses not only other plant homologs of the SSI2 gene, but also using these homologs to engineer enhanced disease resistance or to customize a defense response in other plant species. For example, ssi2 mutant establishes that mutations in this gene result in plants with decreased 18:1 and enhanced resistance to some pathogens. Once the SSI2 homolog of a specific species is isolated, established methods exist to create transgenic plants that are deficient in the SSI2 gene product. These ssi2-like transgenic plants are also considered part of the invention.

Although the ssi2 mutant Arabidopsis exemplified herein is an EMS-induced mutant, any plant may be transgenically engineered to display a similar phenotype. While the natural ssi2 mutant has lost the functional product of the SSI2 gene due to a single point mutation, a transgenic plant can be made that also has a similar loss of the SSI2 product. This approach is particularly appropriate to plants with high ploidy numbers, including but not limited to wheat, corn and cotton. Alternatively, treatment of plants with agents that result in reduction in 18:1 synthesis by, for example, contacting a plant with an agent that inhibits SSI2 transcription, translation, and/or activity; or with an agent that increases ACT1 activity in combination with glycerol administration can be used.

A synthetic mutant can be created by expressing a mutant form of the SSI2 protein or homolog to create a “dominant negative effect”. While not limiting the disclosure to any one mechanism, the mutant SSI2 protein will compete with wild-type SSI2 protein for substrate in a transgenic plant. By over-producing the mutant form of the protein, the signaling pathway used by the wild-type SSI2 protein can be effectively blocked or inhibited. Examples of this type of “dominant negative” effect are well known for both insect and vertebrate systems (Radke et al., 1997, Genetics 145:163-171; Kolch et al., 1991, Nature 349:426-428).

A second kind of synthetic mutant can be created by inhibiting the translation of the SSI2 mRNA by “post-transcriptional gene silencing”. The SSI2 gene or homolog from the species targeted for down-regulation, or a fragment thereof, may be utilized to control the production of the encoded protein. Full-length antisense molecules can be used for this purpose. Alternatively, antisense oligonucleotides targeted to specific regions of the SSI2-encoded RNA that are critical for translation may be utilized. The use of antisense molecules to decrease expression levels of a pre-determined gene are known in the art. Antisense molecules may be provided in situ by transforming plant cells with a DNA construct which, upon transcription, produces the antisense RNA sequences. Such constructs can be designed to produce full-length or partial antisense sequences. This gene silencing effect can be enhanced by transgenically over-producing both sense and antisense RNA of the gene coding sequence so that a high amount of dsRNA is produced (for example see Waterhouse et al., 1998, PNAS 95:13959-13964). In one embodiment, part or all of the SSI2 coding sequence antisense strand is expressed by a transgene. In a particularly preferred embodiment, hybridizing sense and antisense strands of part or all of the SSI2 coding sequence are transgenically expressed.

A third type of synthetic mutant can also be created by the technique of “co-suppression”. Plant cells are transformed with a copy of the endogenous gene targeted for repression. In many cases, this results in the complete repression of the native gene as well as the transgene. In one embodiment, the SSI2 gene or homolog from the plant species of interest is isolated and used to transform cells of that same species.

Transgenic plants with one of the transgenes mentioned above can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994).

The method of transformation depends upon the plant to be transformed. Agrobacterium vectors are often used to transform dicot species. Agrobacterium binary vectors include, but are not limited to, BIN19 (Bevan, 1984) and derivatives thereof, the pBI vector series (Jefferson et al., 1987), and binary vectors pGA482 and pGA492 (An, 1986) For transformation of monocot species, biolistc bombardment with particles coated with transforming DNA and silicon fibers coated with transforming DNA are often useful for nuclear transformation.

DNA constructs for transforming a selected plant comprise a coding sequence of interest operably linked to appropriate 5′ (e.g., promoters and translational regulatory sequences) and 3′ regulatory sequences (e.g., terminators). In one embodiment, the coding region is placed under a powerful constitutive promoter, such as the Cauliflower Mosaic Virus (CaMV) 35S promoter or the figwort mosaic virus 35S promoter. Other constitutive promoters contemplated for use include, but are not limited to, T-DNA mannopine synthetase, nopaline synthase (NOS) and octopine synthase (OCS) promoters.

Transgenic plants expressing a sense or antisense SSI2 coding sequence under an inducible promoter are also contemplated to be within the scope of the disclosure. Inducible plant promoters include the tetracycline repressor/operator controlled promoter, the heat shock gene promoters, stress (e.g., wounding) -induced promoters, defense responsive gene promoters (e.g., phenylalanine ammonia lyase genes), wound induced gene promoters (e.g., hydroxyproline rich cell wall protein genes), chemically-inducible gene promoters (e.g., nitrate reductase genes, glucanase genes, chitinase genes, and the like) and dark-inducible gene promoters (e.g., asparagine synthetase gene) to name a few.

Tissue specific and development-specific promoters are also contemplated for use. Examples of these include, but are not limited to, the ribulose bisphosphate carboxylase (RuBisCo) small subunit gene promoters or chlorophyll a/b binding protein (CAB) gene promoters for expression in photosynthetic tissue; the various seed storage protein gene promoters for expression in seeds; and the root-specific glutamine synthetase gene promoters where expression in roots is desired.

The coding region is also operably linked to an appropriate 3′ regulatory sequence. In one embodiment, the nopaline synthetase polyadenylation region (NOS) is used. Other useful 3′ regulatory regions include, but are not limited to, the octopine (OCS) polyadenylation region.

Using an Agrobacterium binary vector system for transformation, the selected coding region, under control of a constitutive or inducible promoter as described above, is linked to a nuclear drug resistance marker, such as kanamycin resistance. Other useful selectable marker systems include, but are not limited to, other genes that confer antibiotic resistances (e.g., resistance to hygromycin or bialaphos) or herbicide resistance (e.g., resistance to sulfonylurea, phosphinothricin, or glyphosate).

Plants are transformed and thereafter screened for one or more properties, including the lack of SSI2 protein, SSI2 mRNA, constitutive HR-like lesions or expression of PR genes, altered FA metabolism (e.g., reduced 18:1 production) or enhanced resistance to a selected plant pathogen, such as P. parasitica and Pseudomonas. It should be recognized that the amount of expression, as well as the tissue-specific pattern of expression of the transgenes in transformed plants can vary depending on the position of their insertion into the nuclear genome. Such positional effects are well known in the art. For this reason, several nuclear transformants should be regenerated and tested for expression of the transgene.

Transgenic plants that exhibit one or more of the aforementioned desirable phenotypes can be used for plant breeding, or directly in agricultural or horticultural applications. Plants containing one transgene may also be crossed with plants containing a complementary transgene in order to produce plants with enhanced or combined phenotypes.

SSI2 mutants display a unique combination of defense responses that include constitutive HR and expression of PR genes and enhanced disease resistance to certain plant pathogens, and therefore can be used to improve crop and horticultural plant species by customizing the defense response. Plants species contemplated in regard to this disclosure include, but are not limited to, alfalfa, aster, barley, begonia, beet, canola, cantaloupe, carrot, chrysanthemum, clover, corn, cotton, cucumber, delphinium, grape, lawn and turf grasses, lettuce, pea, peppermint, rice, rutabaga, sorghum, sugar beet, sunflower, tobacco, tomatillo, tomato, turnip, wheat, and zinnia.

The SSI2 mutant of Arabidopsis exhibits constitutive activation of an NPR1-independent pathway leading to PR gene expression, and a constitutive HR. It is therefore contemplated that the SSI2 mutants will exhibit broad-spectrum resistance against a wide range of fungal, bacterial and viral pathogens. Such pathogens include, but are not limited to, TCV, P. syrinzgae and P. parasitica.

The SSI2 mutants of the disclosure can be used to identify and isolate additional members of this disease resistance pathway. Mutations that, when combined with ssi2, suppress the SSI2 phenotype, are likely to interact directly with SSI2, or to compensate in some other way for the loss of SSI2 function.

The transgenic plants of the disclosure are particularly useful in conferring a phenotype characterized by reduced 18:1 to many different plant species. In this manner, a host of plant species with enhanced SA-independent defense responses can be made and can be used as breeding lines or directly in commercial operations. Such plants can have uses as crop species, or for ornamental use.

A plant that has had functional SSI2 transgenically depleted should exhibit a defense response profile similar to that of the ssi2 Arabidopsis mutant. A transgenic approach is advantageous because it allows ssi2-phenotype plants to be created quickly, without time-consuming mutant generation, selection, and back-crossing. Transgenically created ssi2-phenotype plants have special utility in polyploid plants, such as wheat, where recessive mutations are difficult to detect.

Methods are provided for protecting a plant from a pathogen comprising applying an effective amount of an agent (e.g., glycerol) that decreases 18:1 or composition of the disclosure to the environment of the pathogen. “Effective amount” is intended to mean an amount of a protein or composition sufficient to control a pathogen. An agent (e.g., glycerol) that decreases 18:1 s and compositions can be applied to the environment of the pathogen by methods known to those of ordinary skill in the art.

The compositions of the disclosure may be obtained by the addition of a surface-active agent, an inert carrier, a preservative, a humectant, a feeding stimulant, an attractant, an encapsulating agent, a binder, an emulsifier, a dye, a UV protective, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pathogens. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present disclosure are normally applied in the form of compositions and can be applied to the crop area, plant, or seed to be treated. For example, the compositions of the present disclosure may be applied during growth, seeding or storage.

The compositions of the present disclosure may be applied simultaneously or in succession with other compounds. Methods of applying an active ingredient of the present disclosure or an agrochemical composition of the present disclosure that contains at least one of an agent (e.g., glycerol) that decreases 18:1 s of the present disclosure include, but are not limited to, foliar application, seed coating, and soil application. The number of applications and the rate of application depend on the intensity of infestation by the corresponding pathogen.

Suitable surface-active agents include, but are not limited to, anionic compounds such as a carboxylate of, for example, a met al; carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include but are not limited to inorganic minerals such as kaolin, phyllosilicates, carbonates, sulfates, phosphates, or botanical materials such as cork, powdered corncobs, peanut hulls, rice hulls, and walnut shells.

The compositions of the present disclosure can be in a suitable form for direct application or as a concentrate of primary composition that requires dilution with a suitable quantity of water or other diluant before application. The concentration of the 18:1 inhibitor will vary depending upon the nature of the particular formulation, specifically, whether it is a concentrate or to be used directly.

A compositions of the disclosure can be applied to the environment of a plant pathogen by, for example, spraying, atomizing, dusting, scattering, coating or pouring, introducing into or on the soil, introducing into irrigation water, by seed treatment or general application or dusting at the time when the pathogen has begun to appear or before the appearance of pathogens as a protective measure. It is generally important to obtain good control of pathogens in the early stages of plant growth, as this is the time when the plant can be most severely damaged. The compositions of the disclosure can conveniently contain an insecticide if this is thought necessary.

Compositions of the disclosure find use in protecting plants, seeds, and plant products in a variety of ways. For example, the compositions can be used in a method that involves placing an effective amount of the composition in the environment of the pathogen by a procedure selected from the group consisting of spraying, dusting, broadcasting, or seed coating.

The methods of the embodiments may be effective against a variety of plant pathogens, such as, but not limited to, Colletotrichum graminocola, Diplodia maydis, Verticillium dahliae, Fusarium graminearum, Fusarium oxysporum and Fusarium verticillioides. Specific pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassuicola, Septoria glycines, Phyllosticta sojicola, Alternaria altemata, Pseudomonas syringae p.v. glycinea, Xanthomonas campestris p.v. phaseoli, Microsphaera diffusa, Fusarium semitectum, Phialophora gregata, Glomerella glycines, Phakopsora pachyrhizi, Pythium aphanidermatum, Pythium ultimum, Pythium debaryanum, Fusarium solani; Canola: Albugo candida, Alternaria brassicae, Leptosphaeria maculans, Rhizoctonia solani, Sclerotinia sclerotiorum, Mycosphaerella brassicicola, Pythium ultimum, Peronospora parasitica, Fusarium roseum, Alternaria alternata; Alfalfa: Clavibacter michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Verticillium albo-atrum, Xanthomonas campestris p.v. alfalfae, Aphanomyces euteiches, Stemphylium herbarum, Stemphylium alfalfae, Colletotrichum trifolii, Leptosphaerulina briosiana, Uromyces striatus, Sclerotinia trifoliorum, Stagonospora meliloti, Stemphylium botryosum, Leptotrichila medicaginis; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, Sunflower: Plasmopora halstedii, Sclerotinia sclerotiorum, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Colletotrichum graminicola, Fusarium verticillioides var. subglutinans, Erwinia stewartii, F. verticillioides, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermaturn, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Claviceps sorghi, Pseudomonas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Sorghum: Exserohilum turcicum, C. sublineolum, Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium verticillioides, Alternaria altemata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

The following examples are intended to illustrate but not limit the invention.

### EXAMPLE

Plants were grown in the MTPS 144 Conviron (Winnipeg, MB, Canada) walk-inchambers at 22° C., 65% relative humidity and 14 hour photoperiod. Crosses were performed by pollinating flowers of Di-17 plants with pollen from Col-0, Nö, ssi2, cpr5, ssi2 act1, ssi2 gly1, act1 and gly1. The double mutant combinations in ssi2 and cpr5 backgrounds were created by crossing HRT ssi2 and HRT cpr5 plants with eds1, sid2, eds5 and pad4. The HRT ssi2 act1 cpr5 plants were derived from a cross between HRT ssi2 act1 and HRT cpr5. The HRT cpr5 act1 plants were derived from a cross between HRT cpr5 and act1. All genotypes were initially screened in the F2 generation and confirmed further in F3 and F4 generations. The genotypes were determined by conducting cleaved amplified polymorphic sequence analysis.

Pathogen inoculations. Transcripts synthesized in vitro from a cloned cDNA of TCV using T7 RNA polymerase were used for viral infections. For inoculations, the viral transcript was suspended at a concentration of 0.05 μg/μl in inoculation buffer, and the inoculation was performed. After viral inoculations, the plants were transferred to a Conviron MTR30 reach-in chamber maintained at 22° C., 65% relative humidity and 14 hour photoperiod. Inoculations with Pseudomonas syringae were conducted.

Fatty acid analysis and oleic acid infiltration. Fatty acid analysis was carried out using gas chromatorgraphy. Oleic acid (Sigma) was dissolved in water and injected into leaves using a needle-less syringe. To determine the 18:1 concentration required to elevate the leaf 18:1 content in ssi2 plants to wild-type levels, 50 μm to 10 mM were injected into ssi2 leaves followed by fatty acid profiling. A concentration of 500 μm to 1 mM was considered optimal, with similar results being obtained upon injection of either of these concentrations.

SA and glycerol treatments. SA and glycerol treatments were carried out by spraying 500 μM and 50 mM solutions, respectively, prepared in sterile water.

RNA extraction, RT-PCR and northern analyses. Small-scale extraction of RNA from one or two leaves was performed in the TRIzol reagent (Invitrogen, Gaihersburg, Md.) following the manufacturer's instructions. RNA quality and concentration were determined by gel electrophoresis and determination of A₂₆₀. Northern blot analysis and synthesis of random primed probes for PR-1 were carried out. Reverse transcription (RT) and first strand cDNA synthesis was carried out using Superscript II (Invitrogen). Two-to-three independent RNA preparations were used for RT-PCR and each of these were analyzed at least twice by RT-PCR. The RT-PCR was carried out for 35 cycles in order to determine absolute levels of transcripts. The number of amplification cycles was reduced to 22-25 in order to evaluate and quantify any differences among transcript levels before they reached saturation.

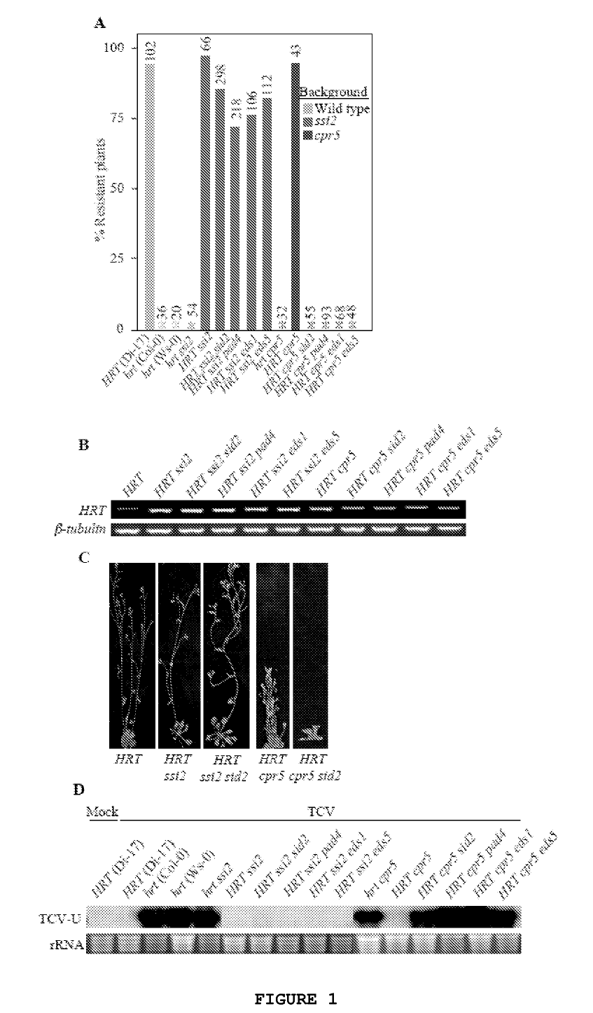

ssi2 mutation upregulates HRT gene expression in an SA- and PAD4-independent manner. To examine the role of high endogenous SA in the upregulation of HRT gene expression, HRT ssi2 and HRT cpr5 plants were mobilized in the sid2 background and obtained HRT ssi2 sid2 and HRT cpr5 sid2 plants. Since sid2 abolishes elevated SA levels in ssi2 and cpr5 plants, a mutation in sid2 was expected to downregulate the expression of HRT and result in susceptibility to TCV. As expected, HRT cpr5 sid2 plants showed basal level expression of HRT and pronounced susceptibility (FIG. 1), confirming that high endogenous SA levels in HRT cpr5 plants were likely responsible for elevated HRT gene expression and thereby enhanced resistance. In contrast to these results, HRT ssi2 sid2 plants continued to show high levels of HRT transcript (FIG. 1B). Consistent with the expression levels of HRT, the HRT ssi2 sid2 plants continued to show heightened resistance to TCV (FIG. 1A, C, D), suggesting that induction of HRT and TCV resistance in HRT ssi2 sid2 plants was independent of SA. The ssi2-mediated induction of HRT was also independent of mutations in eds1, pad4 and eds5, indicating that, in ssi2 plants, a factor(s) other than SA was responsible for the induced expression of HRT. However, as compared to HRT ssi2 plants, the HRT ssi2 sid2, HRT ssi2 eds5, HRT ssi2 eds1 and HRT ssi2 pad4 plants showed ˜15%, ˜18%, ˜20% and ˜25% reductions in the number of resistant plants, respectively (FIG. 1A). This suggests that both SA-dependent and -independent signaling pathways triggered in HRT ssi2 plants have an additive effect on resistance to TCV. In contrast to HRT ssi2 plants, mutations in pad4, eds1 and eds5 abolished both induced expression of HRT as well as resistance to TCV in HRT cpr5 plants (FIG. 1).

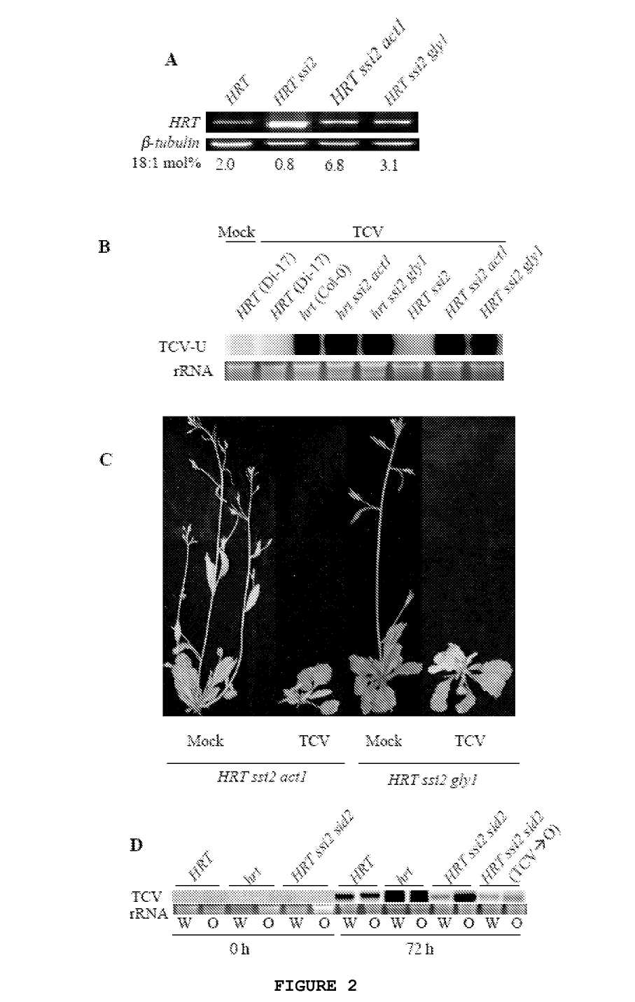

Restoration of 18:1 levels in HRT ssi2 plants compromises resistance to TCV. Since altered defense signaling in the ssi2 plants has been attributed to the reduced levels of 18:1, the restoration of 18:1 levels would be expected to restore the HRT gene expression to wild-type levels. HRT (Di-17 ecotype) plants were crossed with ssi2 act1 and ssi2 gly1 plants; mutations in act1 and gly1 overcome ssi2-triggered phenotypes by increasing the 18:1 content (FIG. 6A). Interestingly, HRT ssi2 act1 and HRT ssi2 gly1 plants showed basal level expression of HRT (FIG. 2A) and only 25% of these plants showed resistance to TCV, which corresponds with the segregation of rrt (Table 1, FIGS. 2B and C). Fatty acid profiling showed that, unlike HRT ssi2 and HRT ssi2 sid2, the HRT ssi2 act1 and HRT ssi2 gly1 plants showed wild-type-like or higher levels of 18:1 (FIG. 2A). These data indicate that restoration of 18:1 levels in HRT ssi2 plants was sufficient to restore the basal level expression of HRT. Control crosses between Di-17 (resistant) and act1 or gly1 (both susceptible) segregated normally for HR and resistance (Table 1), suggesting that these phenotypes were not influenced by mutations in act1 or gly1.

TABLE 1
Epistatic analyses of F2 populations generated by
crossing Di-17 with various wild type or mutant lines.

| | Total | | Number of | | | | | |
|---|---|---|---|---|---|---|---|---|
| | plants | | plants | | | | | |
| Cross | analyzed | Genotypeₐ | obtained | HR_{b} | R_{c} | S_{d} | X² | Pₑ |
| Di-17 × Col-0 | 136 | HRT/ | 93 | + | 18 | 75 | 1.58 | 0.21 |
| Di-17 × Nö | 132 | HRT/ | 94 | + | 22 | 72 | 0.13 | 0.72 |
| Di-17 × ssi2 | 378 | HRT/-SSI2/-ACT1/ | 157 | + | 36 | 121 | 0.36 | 0.55 |
| act1 | | HRT/-ssi2 | 66 | ND | 62 | 4 | 167.2 | 0.00_{f} |
| | | HRT/-act1 | 54 | + | 11 | 43 | 0.62 | 0.43 |
| | | HRT/-ssi2 act1 | 37 | + | 9 | 28 | 0.03 | 0.86 |
| Di-17 × ssi2 | 301 | HRT/-SSI2/-GLY1/ | 108 | + | 26 | 82 | 0.05 | 0.82 |
| gly1–3 | | HRT/-ssi2 | 53 | ND | 51 | 2 | 143.4 | 0.00_{f} |
| | | HRT/-gly1 | 44 | + | 12 | 32 | 0.12 | 0.73 |
| | | HRT/-ssi2 gly1 | 31 | + | 6 | 25 | 0.53 | 0.47 |
| Di-17 × act1 | 123 | HRT/-ACT1/ | 84 | + | 19 | 65 | 0.25 | 0.62 |
| | | HRT/-act1 | 12 | + | 3 | 9 | 0.00 | 1.00 |
| Di-17 × gly1–3 | 126 | HRT/-GLY1/ | 71 | + | 16 | 55 | 0.23 | 0.63 |
| | | HRT/-gly1 | 22 | + | 8 | 14 | 1.51 | 0.22 |

ₐThe genotype at HRT and various mutant loci was determined by CAPS analysis.
_{b}HR, Hypersensitive response.
_{c}Resistant.
_{d}Susceptible.
ₑOne degree of freedom.
_{f}Statistically significant.
ND Not determined; these plants show spontaneous HR.

To determine if exogenous application of 18:1 in HRT ssi2 plants had the same effect as the act1 or gly1 mutations, Di-17, Col-0 (susceptible) and HRT ssi2 sid2 plants were infiltrated with 18:1 (FIG. 2D). Injection of 18:1 did not appear to alter TCV replication in the inoculated leaves of wild-type plants; similar levels of TCV transcript were detected in water- and 18:1-treated Di-17 or Col-0 leaves. As expected, the susceptible Col-0 plants supported increased replication of the virus; TCV transcript levels in the inoculated leaf were several folds higher than in the resistant Di-17 plants. The inoculated leaves of HRT ssi2 sid2 plants accumulated 2-3 fold lower TCV transcript than Di-17, suggesting that these plants supported reduced viral replication as compared to Di-17. Strikingly, infiltration of 18:1 into HRT ssi2 sid2 leaves increased the amount of TCV transcript by ˜6-8 fold. However, TCV replication remained unaffected if 18:1 was infiltrated 24 h after TCV inoculation (FIG. 2D, last two lanes), indicating that 18:1 levels during the initial stages of pathogen perception were crucial for resistance signaling against TCV. Interestingly, although 18:1 injections increased TCV transcript at the localized site of inoculation, it did not allow systemic spread of the virus in HRT ssi2 sid2 plants. This could be because constitutive expression of HRT is likely to initiate downstream events leading to resistance, and a temporary surge in 18:1 levels at the localized site of infiltration may not be sufficient to promote systemic spread of the virus. Indeed, exogenous application only normalized 18:1 levels locally, at the site of infiltration, while the remaining untreated leaves continued to show low levels of 18:1. The above assumption is further supported by the results related to light-mediated resistance signaling to TCV, which showed that dependence on light can be overcome by initiating downstream signaling prior to dark treatment. Thus, exogenous application of SA prior to dark-treatment of Di-17 plants or the presence of the ssi2 or cpr5 mutations in HRT plants prevents dark-triggered susceptibility.

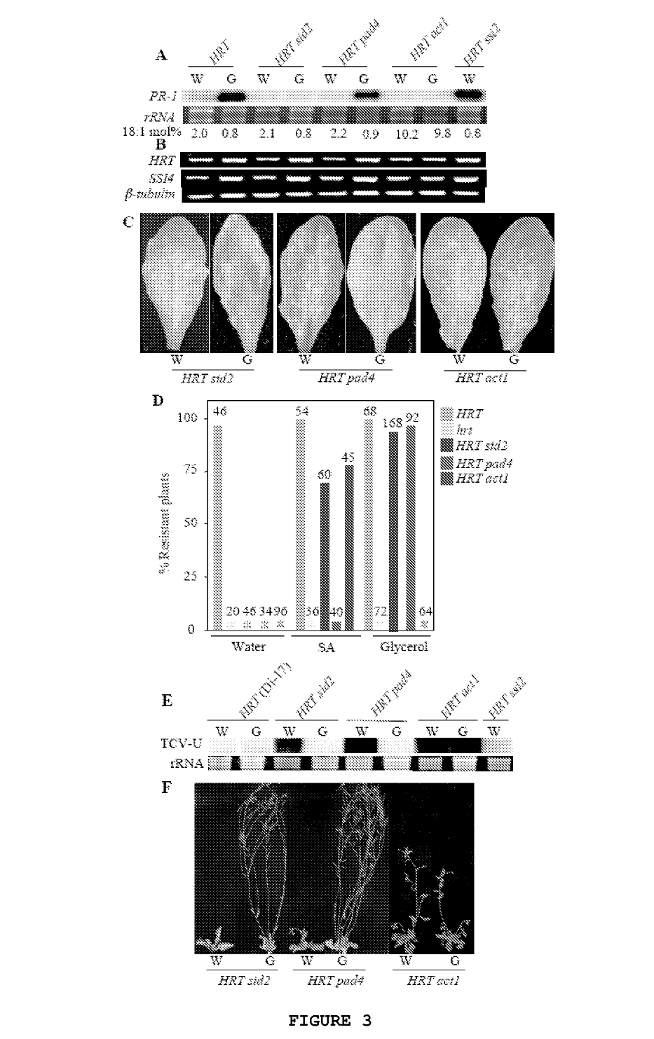

Exogenous application of glycerol confers resistance to TCV in an SA-independent but ACT1-dependent manner. Additional evidence supporting a role for 18:1 in regulating HRT gene expression was obtained by glycerol treatment of several genotypes. Exogenous application of glycerol lowered 18:1 content in wild-type, sid2 and pad4 backgrounds but not in act1 plants. This is because the act1 plants are blocked in the step leading to the acylation of glycerol-3-phosphate with 18:1. In addition, glycerol application induced high levels of SA, and that this induction was dependent on the presence of a functional SID2 protein. As predicted, exogenous application of glycerol lowered 18:1 content in wild-type, HRT sid2 and HRT pad4, plants but not in HRT act1 plants (FIG. 2C). Consequently, wild-type and HRT pad4 plants showed upregulation of PR-1 gene expression as opposed to HRT act1 plants (FIG. 3A). HRT sid2 plants failed to induce PR-1 gene expression in response to exogenous glycerol due to their inability to increase SA levels. Most importantly, glycerol-mediated reduction in 18:1 levels induced HRT transcript levels in wild-type, HRT sid2 and HRT pad4, but not in HRT act1, plants (FIGS. 3A and B). Since the upregulation of HRT abolishes visible HR to TCV, the development of HR in various glycerol-treated plants was monitored. With the exception of HRT act1 plants, all other genotypes, including wild-type, HRT sid2 and HRT pad4 plants, developed no visible HR in response to glycerol (FIG. 3C). Thus, although the SA-mediated induction of HRT is dependent on PAD4, 18:1-mediated induction of HRT is independent of PAD4. The glycerol bioassay further confirmed that HRT expression can be induced in an SA-independent manner by lowering the levels of 18:1 and that this effect was specific because act1 plants failed to induce HRT upon glycerol application.

Consistent with the HRT transcript levels, exogenous application of glycerol significantly enhanced resistance in HRT pad4 plants to wild-type levels (FIG. 3D, E, F). By contrast, pretreatment of HRT pad4 plants with SA resulted in only a marginal increase in the percentage of resistant plants. Similarly, glycerol treatment also enhanced resistance to TCV in HRT sid2 plants, but not in HRT act1 or Col-0 plants. However, SA pretreatment of HRT act1 plants led to a substantial increase in the percentage of TCV-resistant plants, suggesting that SA- and glycerol-triggered pathways were mutually exclusive. This was further confirmed by generating HRT cpr5 act1 and HRT ssi2 act1 cpr5 plants; unlike HRT ssi2 act1 plants, resistance in HRT cpr5 act1 and HRT ssi2 act1 cpr5 was comparable to that seen in HRT cpr5 plants. Taken together, these data suggest that there are at least two mechanisms leading to the induction of HRT, one dependent on SA and PAD4 and the other responsive to reduced levels of 18:1. Furthermore, these data suggest that glycerol treatment is more effective in conferring resistance; in comparison to SA, pretreatment with glycerol produced ˜30% more resistant HRT sid2 plants.

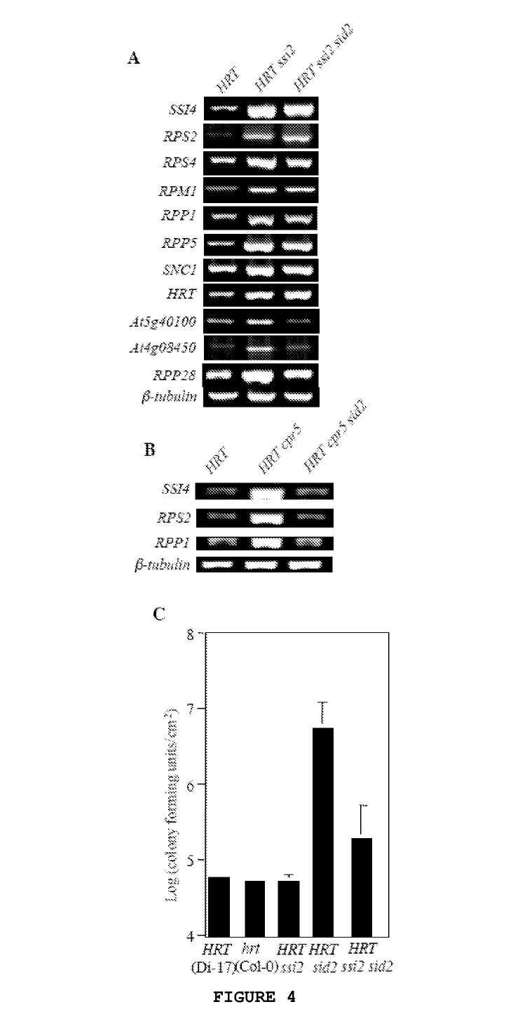

Low 18:1 conditions upregulate expression of structurally different R genes. In order to examine if the SA-independent and 18:1-modulated induction was exclusive to HRT, the expression of several other R genes in the ssi2 background (FIG. 4A) were analyzed. Some of the R genes (At5g40100, At4g08450, RPP28) were induced in ssi2 plants but showed basal level expression in HRT ssi2 sid2 plants. However, many of the NB-LRR genes, encoding proteins containing either coiled coil (such as HRT, RPS2, RPM1) or Toll-interleukin-1-like (RPS4, RPP1, RPP5, SSI4, SNC1) domains at their N-terminal, showed elevated transcript level in both HRT ssi2 and HRT ssi2 sid2 backgrounds. By contrast, HRT ssi2 act1 and HRT cpr5 sid2 plants showed basal level expression of all the R genes that were upregulated in HRT ssi2 sid2 plants (FIG. 4B). These data suggest that induction of R genes in HRT ssi2 sid2 plants was dependent on low 18:1 levels but independent of SA. In addition, these results suggest that high SA level in ssi2 and cpr5 background was responsible for increasing transcript level of several R genes.

To determine if upregulation of R genes in the HRT ssi2 sid2 background also conferred SA-independent disease resistance to non-viral pathogens, wild-type, HRT sid2, HRT ssi2 and HRT ssi2 sid2 plants (containing RPS2) were inoculated with an avirulent isolate of the bacterial pathogen Pseudomonas syringae (containing AvrRpt2). The resistance mediated by RPS2 is dependent on SA. As expected, HRT sid2 plants showed severe susceptibility whereas HRT ssi2 and wild-type plants (Di-17, Col-0 or Nössen) showed resistance. Interestingly, although HRT ssi2 sid2 plants showed increased susceptibility as compared to HRT ssi2 or wild-type plants, they showed an ˜70-fold decrease in bacterial titer as compared to HRT sid2 plants. This suggests that SA-independent enhanced resistance, mediated by reduced 18:1 levels, was not specific to a certain group of pathogen and was conferred due to increased expression of the corresponding R genes. Since the SA-depleted ssi2 nahG plants have previously been shown to display enhanced resistance to virulent pathogens and insects, the SA-independent induction of multiple R genes is also likely to contribute to generalized defense in these plants.

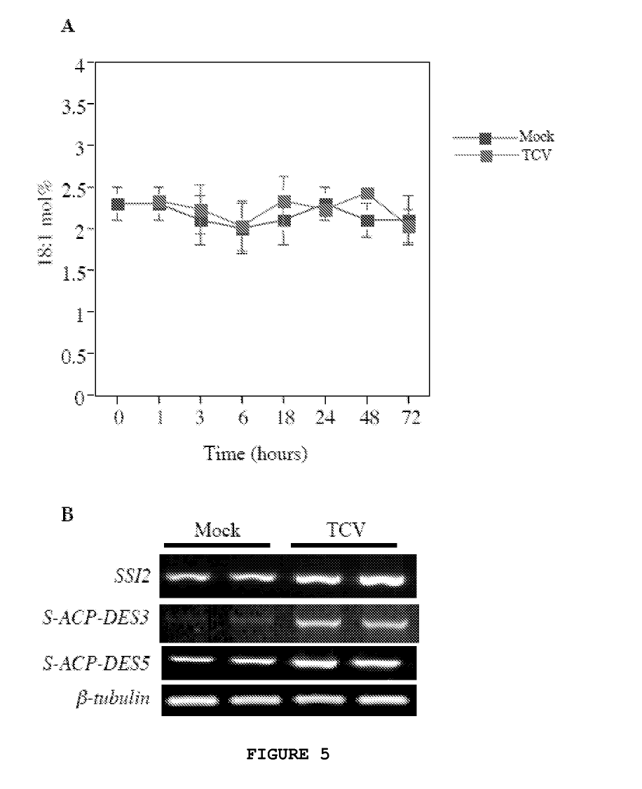

The disclosure provides a novel fatty acid-dependent signaling pathway which, when activated, can confer broad-spectrum resistance to pathogens. Intriguingly, 18:1 levels did not change during a resistant response to TCV (FIG. 5A). Furthermore, TCV inoculation led to the induction of SSI2 and two other stearoyl-acyl carrier protein desaturases, all of which are capable of synthesizing 18:1 (FIG. 4B). Since 18:1 levels in plants are under post-transcriptional and post-translational controls, induction of various desaturases is unlikely to increase 18:1 above wild-type levels but is likely to prevent a decline in the 18:1 content. Since SA-dependent and 18:1-regulated pathways have additive effects, it is possible that pathogens have developed mechanisms to shut-off one or more defense pathway(s) to increase their chances of survival in the host.

Although the invention has been described with reference to the examples above, it should be understood that various modifications can be made without departing from the spirit of the invention.

What is claimed is:

1. A method of inducing pathogen resistance in a plant comprising contacting the plant with an agent that reduces 18:1 fatty acids, wherein the reduction of 18:1 fatty acids increases the production of resistance gene transcription.

2. The method of claim 1, wherein the agent comprises a polynucleotide that reduces or inhibits the function of a sterayl-acyl carrier protein desaturase.

3. The method of claim 2, wherein reduction or inhibition of function of the desaturase in a plant results in altered resistance of the plant to plant pathogens or other disease-causing agents and/or insect tolerance.

4. The method of claim 2, wherein reduction or inhibition of function of the desaturase causes a reduction in 18:1 fatty acids and an SA-independent constitutive expression of PR genes.

5. A method of inducing pathogen resistance in a plant comprising contacting the plant with a composition that reduces or inhibits the production of 18:1 and causes increased transcription of resistance genes.

6. The method of claim 5, wherein the composition comprises glycerol or a glycerol derivative.

7. The method of claim 5, wherein the composition comprises salicylic acid, and glycerol or a glycerol derivative.

8. The method of claim 6, further comprising increasing the transcription of act1 or the activity of ACT1.

9. A method of increasing HRT gene expression in a plant comprising inhibiting the production of 18:1.

10. The method of claim 9, wherein the inhibition is by contacting the plant with glycerol or a glycerol derivative.

11. The method of claim 9, wherein the inhibition is by inhibiting the activity or production of SSI2 or a stearoyl-acyl carrier protein desaturase.

12. The method of claim 11, wherein the production of SSI2 is inhibited by an siRNA or ribozyme.

13. The method of claim 11, wherein the activity of SSI2 is inhibited by an antibody the specifically binds to SSI2.

14. A transgenic plant that is resistant to a pathogen comprising reduced expression of SSI2, wherein the plant comprises reduced 18:1.

15. The transgenic plant of claim 14 comprising an ssi2 mutation.

16. The transgenic plant of claim 14, wherein the plant comprises an antisense polynucleotide to SSI2.

17. The transgenic plant of claim 14, comprising an increase in 18:0 fatty acids and a reduction in 18:1 fatty acids.

18. A method to enhance resistance of a plant to plant pathogens or other disease causing agents, comprising decreasing production or activity of a SSI2 gene product in the plant.

19. The method of claim 18, wherein the SSI2 production or activity is increased by the addition of at least one transgene to the plant genome.

20. A fertile plant produced by the method of claim 18.
